(19) United States
(12) Patent Application Publication
(10) Pub. No.: US 2025/0017523 A1
(43) Pub. Date: Jan. 16, 2025

(54) BRUXISM DETECTION AND FEEDBACK SYSTEM AND METHOD

(71) Applicant: JAWSAVER B.V., UTRECHT (NL)

(72) Inventors: Gabriel Enrique SAENZ, UTRECHT (NL); Selina TIRTAJANA, UTRECHT (NL); David Benjamin JAROCH, ARVADA, CO (US)

(21) Appl. No.: 18/715,138

(22) PCT Filed: Nov. 29, 2022
(86) PCT No.: PCT/EP2022/083567

§ 371 Date: May 31, 2024

(30) Foreign Application Priority Data
Dec. 1, 2021 (NL) 2029986

(51) Int. Cl. A61B 5/00; A61B 5/0205; A61B 5/024; A61B 5/08

(52) U.S. Cl. CPC A61B 5/4557; A61B 5/0205; A61B 5/486; A61B 5/6817; A61B 5/02438; A61B 5/0816; A61B 2562/0204

(57) ABSTRACT

The invention relates to a bruxism detection and feedback system comprising an in-ear device having an in-ear portion and a feedback device. The in-ear device comprises a heart rate sensor and/or a breathing rate sensor and a bruxism sensor. The bruxism detection and feedback device further comprises a processor connected to the heart rate sensor and/or the breathing rate sensor, the bruxism sensor and the feedback device. In embodiments the bruxism sensor is an acoustic sensor for measuring an acoustic signal in the ear canal of a wearer. The invention further relates to a method for use of the bruxism detection and feedback system.

### CROSS-REFERENCE TO RELATED APPLICATIONS

This application is the National Stage of International Application No. PCT/EP2022/083567, filed Nov. 29, 2022, which claims priority to NL 2029986, filed Dec. 1, 2021, all of which are entirely incorporated herein by reference.

### FIELD OF THE INVENTION

The invention relates to a bruxism detection and feedback system comprising an in-ear device having an in-ear portion and a feedback device. The invention further relates to a method for use of the bruxism detection and feedback system.

### BACKGROUND OF THE INVENTION

Bruxism may be characterized by repetitive jaw-muscle activity which leads to clenching or grinding of the teeth and/or bracing and thrusting of the mandible. Bruxism is often caused at a subconscious level wherein a person suffering from bruxism is not aware that a bruxism episode or bruxism event is happening. Bruxism may occur during all moments of the day, e.g. during sleep or when the person is awake. Different persons may suffer from bruxism in different intensities.

Bruxism may lead to related issues such as dental issues, headaches, facial and jaw pain and a sore neck. These issues may be associated with cumulative damage imposed on the teeth and/or the temporomandibular joint (TMJ).

Biofeedback, or feedback, has been shown to be a potential tool in managing bruxism. By providing feedback (external stimulus) during a bruxism event, e.g. through nudging or alerting a person, the person may modify their subconscious behaviour and reduce or stop the bruxism event. Repeated use of feedback may create a learned response in the person that persists even after cessation of the feedback.

KR20200065357A discloses an apparatus for measuring bruxism and a system for diagnosing bruxism. The apparatus for measuring bruxism may be worn in the ear in the form of an earphone and provides a signal to an earphone wearer when a bruxism event is occurring, allowing the wearer to become conscious of the event and to stop the event.

The use of an in-ear device compared to a mouth guard or sensors worn on the face with a strap is advantageous because the in-ear device is less intrusive and may be worn throughout the day and night without being obvious to an observer.

A downside of the use of an in-ear device for measuring bruxism is that the in-ear device has to be relatively small to be able to fit in an ear canal and be supported by the ear canal. In particular, an in-ear device that is worn during sleep may not extend too far out of the ear canal to not be uncomfortable. Additionally, in order to use the in-ear device during the day, e.g. at work, the in-ear device may also not extend too far out of the ear for it to be obvious to an observer that it is worn.

A factor in determining the size of the in-ear device is the local power source of the in-ear device. Preferably, the bruxism detection and feedback system is able to detect and provide feedback for a longer amount of time before needing to be recharged. A larger local power source of the in-ear device may increase the amount of time needed before recharging but may also increase the size of the in-ear device.

### OBJECT OF THE INVENTION

An object of the first aspect of the invention is to provide a bruxism detection and feedback system that allows for accurate detection of bruxism events while simultaneously being power efficient.

### SUMMARY OF THE INVENTION

This object is achieved with the bruxism detection and feedback system of the first aspect comprising:
an in-ear device comprising:
   An in-ear portion configured to be placed in an ear canal, wherein preferably the in-ear portion is at least partially made from a shape conforming material that conforms to a shape of the ear canal;
   A heart rate sensor to measure a heart rate, a microvibration sensor to measure microvibrations and/or a breathing rate sensor to measure a breathing rate when the in-ear portion is placed in the ear canal; and
   a bruxism sensor configured for measuring a characteristic signal which is characteristic of a bruxism event when the in-ear portion is placed in the ear canal,
a processor connected to the heart rate sensor, the microvibration sensor and/or the breathing rate sensor for receiving the measured heart rate, the measured microvibrations and/or measured breathing rate, and connected to the bruxism sensor for receiving the measured characteristic signal; and
a feedback device connected to the processor, wherein the feedback device is configured to provide a feedback stimulus when the feedback device receives a bruxism event signal from the processor,
   wherein the processor is configured to
determine the occurrence of a microarousal event by comparing the measured heart rate, the measured microvibrations and/or the measured breathing rate to a target heart rate and/or a target breathing rate;
increase a measuring rate of the bruxism sensor when the occurrence of the microarousal event is determined by the processor;
receive the characteristic signal measured by the bruxism sensor;
determine the occurrence of a bruxism event by comparing the characteristic signal measured at the increased measuring rate to a target characteristic; and
send the bruxism event signal to the feedback device when the processor determines the occurrence of the bruxism event.

The TMJ is located close to the ear canal, such that movement of the TMJ deforms the shape of the ear canal. In particular movement of the TMJ leads to bending of the walls of the ear canal. During jaw movements, such as chewing and during bruxism events, the TMJ moves and changes the shape of the ear canal. The movement of the TMJ depends on the jaw movement and differs between normal jaw movements, such as chewing or talking, and between bruxism events. This difference in movement translates in different changes of shape in the ear canal. This allows a bruxism event to be detected in the ear canal by an in-ear device.

The bruxism detection and feedback system of the first aspect of the invention comprises an in-ear device with an in-ear portion that is configured to be placed in an ear canal of a wearer. The in-ear portion may be made from a shape conforming material that conforms to the shape of the ear canal. For example, the in-ear device, and in particular the in-ear portion, may be personalized to fit into the ear canal of an individual user. This may be done using standard fitting techniques. In use, the in-ear portion may support the in-ear device in the ear of the wearer.

The in-ear device further comprises a heart rate sensor to measure a heart rate, a microvibration sensor to measure microvibrations and/or a breathing rate sensor to measure a breathing rate when the in-ear portion is placed in the ear-canal. The heart rate sensor, the microvibration sensor and the breathing rate sensor may be provided in the in-ear portion of the device. In embodiments the heart-rate sensor may acquire measurements related to the heart rate of the wearer at a frequency below 5 Hz, preferably below 3 Hz. In embodiments the breathing rate sensor acquires measurements related to the breathing rate of the wearer at a frequency between 0.05 Hz and 0.5 Hz, for example between 0.07 Hz and 0.33 Hz.

The in-ear device further comprises a bruxism sensor configured for measuring a characteristic signal which is characteristic of a bruxism event when the in-ear portion is placed in the ear canal. The bruxism sensor may be completely or partly provided in the in-ear portion of the in-ear device. As explained above, jaw movement causes movement of the TMJ which influences the shape of the ear canal. Different jaw movements, related to different activities, influence the shape of the ear canal in different ways. The bruxism sensor is configured for directly or indirectly measuring the effect of the TMJ on the shape of the ear canal. For example, the bruxism sensor may be an acoustic sensor for measuring acoustic signals in the ear canal caused by the changes in shape of the ear canal. In another example, the bruxism sensor may be a strain gauge which comprises a wire grid provided in the in-ear portion of the in-ear device. Changes in the shape of the in-ear portion caused by changes in the shape of the ear canal change the resistances of the wire grid which may be measured. In another example, the bruxism sensor comprises magnets embedded in the in-ear portion to create a known electromagnetic (EM) field in the ear canal that changes upon changes in shape of the ear canal. Other embodiments of the bruxism sensor are possible such as an optical sensor or a piezoelectric sensor.

The bruxism detection and feedback system further comprises a processor connected to the heart rate sensor and/or the breathing rate sensor for receiving the measured heart rate and/or the measured breathing rate. The processor is further connected to the bruxism sensor for receiving the measured characteristic signal. In embodiments the processor is provided in the in-ear device. In other embodiments the processor is provided in a separate device, e.g. the processor is part of a smart device, such as a smart phone. In embodiments the processor is provided in the feedback device, e.g. in the form of a processor in a wristband. The processor and the feedback device may both also be provided in a smart device, e.g. in a smart phone or a smart watch. The processor may use an algorithm, e.g. artificial intelligence (AI) techniques, such as reinforcement learning, to improve the detection of bruxism events, microarousal events, or the effect of feedback stimuli. The processor may further be configured to send a microarousal event signal to the feedback device such that the feedback device provides a feedback stimulus to pre-empt the onset of the bruxism event.

The bruxism detection and feedback system further comprises a feedback device connected to the processor. The feedback device is configured to provide a feedback stimulus, e.g. a biofeedback stimulus, to the wearer, when the feedback device receives a bruxism event signal from the processor. The feedback devices may be embodied different ways. The feedback device may also be multiple feedback devices or a single feedback device capable of providing several distinct feedback stimuli. The feedback may be a strong feedback stimulus that consciously alerts the wearer, and the feedback may be a weak feedback stimulus that unconsciously alerts the wearer, e.g. a feedback stimulus that stops the bruxism event but does not wake the wearer. The feedback device may also be provided in the in-ear device, e.g. in the form of a microphone, a vibration motor, or an electrical stimulator.

The processor is configured to determine the occurrence of a microarousal event by comparing the measured heart rate, the measured microvibrations and/or the measured breathing rate to a target heart rate, target microvibrations and/or a target breathing rate. For example, the target heart rate, target microvibrations and/or the target breathing rate may be a baseline heart rate, microvibrations and/or breathing rate of the wearer, so that the target heart rate, the target microvibrations and/or the target breathing rate are personalized. The occurrence of the microarousal event may be determined when the measured values rapidly change from the baseline values. In another example the occurrence is determined when the measured values exceed a target value, which may be determined based on the baseline values. In another example, the occurrence may be determined when the measured values coincide with the target values.

The processor is further configured to increase the measuring rate of the bruxism sensor when the occurrence of the microarousal event is determined by the processor. For example, the measuring rate of the bruxism sensor in a lower measuring rate mode may be four times lower than the measuring rate in a high measuring rate mode, which is when the processor increases the measuring rate. In another example, the measuring rate of the bruxism sensor is increased from a zero measurement rate, e.g. the bruxism sensor starts measuring when the occurrence of a microarousal event is determined. The bruxism detection and feedback system may have a standby mode, which is active when no microarousal event is determined, wherein the system measures for the occurrence of a microarousal event and an awake mode, which is triggered when the occurrence of the microarousal event is determined, in which awake mode the measurement rate of the bruxism sensor is increased compared to the measurement rate of the bruxism sensor in the standby mode.

The processor is further configured to determine the occurrence of the bruxism event by comparing the characteristic signal measured by the bruxism sensor to a target characteristic. For example, the bruxism event may be determined to occur by the processor when the measured characteristic signal coincides with the target characteristic. For example, the bruxism sensor may be a strain gauge for measuring bending of the ear canal. When the strain gauge measures a certain bending of the ear canal over a certain period, and this coincides with a target bending and target period, the processor may determine that a bruxism event is occurring. In another example the processor determines the occurrence of the bruxism event when the measured characteristic signal exceeds the target characteristic.

The processor is further configured to send the bruxism event signal to the feedback device when the processor determines the occurrence of the bruxism event. For example, the processor may send multiple bruxism event signals when the feedback sent by the feedback device has not stopped the bruxism event. The feedback stimuli may depend on an intensity of the bruxism event.

The bruxism feedback and detection system allows for accurate detection of the bruxism event by measuring for the characteristic signal with the bruxism sensor. It was found that a bruxism event is preceded by a microarousal event that occurs 5-15 seconds prior to the bruxism event. Thus the bruxism sensor only has to sense for a bruxism event in a period around the occurrence of a microarousal event. For example, the measurement rate of the bruxism sensor is increased for a set period, for example of 30 seconds, when a microarousal is determined. Measuring of breathing rate and/or heart rate may be more energy efficient than measuring for a bruxism event. Additionally, the measuring rate of the breathing rate sensor and/or the heart rate sensor may be lower than the measuring rate of the bruxism sensor, making measuring for a microarousal event more energy efficient than measuring for a bruxism event. In this way the first aspect of the invention allows for a more energy efficient bruxism detection and feedback system.

The bruxism feedback and detection system further allows to reduce the number of false positive bruxism detections by correlating the occurrence of a microarousal event with a higher likelihood of a bruxism episode occurring.

In an embodiment the bruxism sensor is an acoustic sensor for measuring an acoustic signal, e.g., in a time-frequency-amplitude domain, when the in-ear portion is placed in the ear canal, wherein the occurrence of the bruxism event by the processor is determined by comparing the measured acoustic signal, e.g. in the measured time-frequency-amplitude domain, to a target acoustic signal, e.g. in a target time-frequency-amplitude domain.

Movement of the TMJ results in changes of shape in the ear canal which leads to acoustic signals, e.g., sound waves, in the ear canal which may be measured by the acoustic sensor. The acoustic signals contain information on the specific changes in shape occurring in the ear canal. Thus, by measuring the acoustic signals information on bruxism may be obtained.

For example, the acoustic sensor is responsive to a broad band of acoustic signals, e.g. audible and inaudible sounds. For example, the acoustic signal has a frequency of less than 5000 Hz, for example between 0.1 Hz and 5000 Hz, for example less than 3400 Hz, for example between 100 Hz and 3400 Hz. The acoustic signal may be represented in a time-frequency-amplitude domain which representation may be compared to a target signal in a time-frequency-amplitude domain. For example, when the target signal coincides or has sufficient correlation with the measured signal, the processor determines the occurrence of a bruxism event. In another example, when a measured amplitude and frequency of the acoustic signal exceed or coincide or have sufficient overlap with a target amplitude and a target frequency of a target acoustic signal, does the processor determine the occurrence of a bruxism event.

When no microarousal event has been determined to occur, the acoustic sensor may have a measurement rate with a frequency below 10 kHz, e.g. below 5 kHz, e.g. below 1 kHz. When the microarousal event has been determined to occur, the acoustic sensor may have a measurement rate above 10 kHz. The acoustic sensor may further be used to identify other jaw movements such as chewing or it may be used to distinguish between different types of bruxism events, e.g. between clenching and grinding.

In further embodiments the heart rate sensor, the microvibration sensor and/or the breathing rate sensor are formed by the acoustic sensor. Heart rate, microvibrations and breathing of the wearer results in acoustic signals in the ear canal, e.g. as a result of blood or air flow. Thus, the acoustic sensor used for measuring the occurrence of the bruxism event may also be used for measuring the heart rate, microvibrations and/or the breathing rate. This allows the in-ear device to be smaller and/or more affordable to the end user.

In embodiments, the bruxism sensor, heart rate sensor, microvibration sensor, and/or the breathing rate sensor are formed by an accelerometer. Heart rate, microvibrations and breathing of the wearer results in small movements in the ear canal, e.g. as a result of blood or air flow or low-amplitude muscle movements. Thus, the accelerometer may be used for measuring the heart rate, microvibrations and/or the breathing rate. This allows the in-ear device to be smaller, energy-efficient, and/or more affordable to the end user.

In embodiments, the in-ear device further comprises a flexible resonator provided in the in-ear portion, wherein the resonator is configured to vibrate at a resonance frequency when a resonance sound is emitted, wherein the resonator is connected to the in-ear portion such that, upon a change of shape of the ear canal, the resonance frequency of the resonator changes, e.g. by a deformation of the resonator, wherein the in-ear device further comprises a sound emitter for emitting an excitation signal to excite the resonator, and wherein the bruxism sensor is configured to measure a vibration of the resonator caused by the excitation signal.

For example, the resonator may be a flexible strand of material, such as a string, that is connected on either side of the in-ear portion. Deformation of the ear canal may lead to a deformation of the in-ear portion which changes the resonance frequency of the resonator. By emitting an excitation signal the resonator is excited, which results in a vibration of the resonator. The vibration depends on the resonance frequency of the resonator which depends on the shape of the ear canal. Thus, by measuring the vibration resulting from the resonator information on the shape of the ear canal may be obtained. The change of frequency of the vibration as a result of a fixed excitation signal is proportional to the amount of deformation of the ear canal.

The excitation signal may be a periodic discrete signal at a fixed frequency, but it may also be a periodic continuous signal over a frequency range, or even a non-periodic signal. The vibration may be measured by an acoustic sensor which hears the acoustic signal emitted by the resonator. The vibration may also be measured by a force transducer provided near or at the resonator.

The resonator may be formed by multiple flexible strands of material, e.g. by multiple strings, with different orientation to increase the information. The resonator may comprise multiple resonators having different resonator frequencies or located along different points of the in-ear portion to provide information on changes in shape of different portions of the ear canal. The resonator may further be a ring resonator having a ring like structure or a diaphragm resonator having a closed circle structure.

In embodiments, the in-ear device further comprises a sealing portion to seal the ear canal when the in-ear portion is placed in the ear canal. This embodiment is particularly advantageous when the bruxism sensor is an acoustic sensor as it allows for improved measurements of acoustic signals in the ear canal. This embodiment may also be advantageous when a resonator is used, to prevent the resonator from interacting with excitation signals originating from an outside source.

In embodiments, the in-ear portion comprises an impermeable pouch that conforms to a shape of the ear canal and wherein the bruxism sensor is provided in the impermeable pouch for measuring the characteristic signal in a medium filling the pouch. In this embodiment a change in the shape of the ear canal results in a change of shape of the pouch, which has an effect on the medium filling the pouch. By measuring the medium, e.g. water, air, or another fluid, the change on the shape of the ear canal may be inferred.

In a further embodiment, the pouch is filled with an incompressible fluid. For example, air is understood to be an incompressible fluid.

In embodiments the in-ear device is personalized such that the in-ear portion extends to the second bend of the ear canal when the in-ear portion is placed in the ear canal. This may allow the system to acquire accurate information on the shape of the ear canal, and may also allow the in-ear device to be stably supported by the in-ear portion. In this or other embodiments the in-ear device may be fully contained in the ear canal when worn.

In embodiments, the processor is further configured to determine when the bruxism event terminates, for example by determining when the characteristic signal stops coinciding with the target characteristic. The processor may further determine when the bruxism event terminates when the characteristic signal drops below the target characteristic or when the characteristic signal no longer has a certain correlation to the target characteristic. This allows the processor to send a signal to the feedback device to stop providing feedback. This may further allow the processor to reduce the measurement rate of the bruxism sensor, e.g. to a measurement rate before the occurrence of the microarousal event was determined, or e.g. such that the system returns to a standby mode.

In embodiments the processor is configured to determine characteristics of the bruxism event, for example the processor may determine the duration of the bruxism event, the intensity of the bruxism event, or the type of bruxism event.

In embodiments, the in-ear portion is at least partially made from a shape conforming material and wherein the shape conforming material is one of a body heat-activated memory foam and a silicone, e.g. a moldable silicone.

In embodiments, an intensity, frequency, pattern and/or type of the feedback stimuli is determined by using a reinforcement learning algorithm. Each person may react differently to the biofeedback stimuli. For some wearers a strong biofeedback stimuli is required, while for others the feedback stimuli may be weaker. By allowing reinforcement learning, for example continuous reinforcement learning, the algorithm may deliver and track the effect of varying feedback stimuli on the characteristic of each bruxism event and may identify the most suitable intensity, frequency, pattern and/or type of feedback stimuli in reducing a characteristic of the bruxism event, such as the duration and/or intensity of the event. In this way the feedback stimuli is personalized and dynamic, which prevents adaptation to the feedback stimuli.

In embodiments, the feedback stimuli is one or more of a haptic feedback, an acoustic signal, a mechanical stimulus and an electrical stimulus. An example of a haptic feedback is a vibration.

In embodiments, the processor is further configured to determine a type of bruxism event and wherein the processor is configured to determine a type of feedback stimulus based on the type of the bruxism event. A type of the bruxism event may be a duration of the bruxism event or an intensity of the bruxism event, or whether the bruxism event comprises clenching or grinding. More intense or longer bruxism events may require a stronger feedback stimuli to be provided by the feedback device. This embodiment allows for personalized feedback, e.g. when reinforcement learning is used by the processor to determine for a user a suitable feedback stimuli depending on the type of the bruxism event.

The first aspect of the invention further relates to a method for detecting of bruxism wherein use is made of a bruxism detection and feedback system according to the first and/or second aspects of the invention.

In embodiments the method according to the first aspect comprises:
placing the in-ear portion in the ear canal;
measuring a heart rate using the heart rate sensor, microvibrations using the microvibration sensor, and/or a breathing rate using the breathing sensor;
determining the occurrence of a microarousal event by comparing the measured heart rate, the measured microvibrations, and/or the measured breathing rate to the target heart rate and/or the target breathing rate;
increasing a measuring rate of the bruxism sensor when the occurrence of the microarousal event is determined by the processor
receiving the characteristic signal measured by the bruxism sensor; and
determining the occurrence of a bruxism event by comparing the characteristic signal measured at the increased measuring rate to a target characteristic.

In embodiments the method comprises:
placing the in-ear portion in the ear canal;
measuring the characteristic signal using the bruxism sensor;
determining, by the processor, the occurrence of the bruxism event by comparing the characteristic signal to the target characteristic;
sending, by the processor, the bruxism event signal to the feedback device when the processor determines the occurrence of the bruxism event; and
providing a feedback stimuli by the feedback device.

The second aspect of the invention relates to a bruxism detection and feedback system for more accurately measuring the occurrence of a bruxism event.

The bruxism detection and feedback system according to the second aspect of the invention comprises:
an in-ear device comprising:
   an in-ear portion configured to be placed in an ear canal, wherein preferably the in-ear portion is at least partially made from a shape conforming material that conforms to a shape of the ear canal; and
   an acoustic sensor for measuring an acoustic signal comprising an amplitude and a frequency when the in-ear portion is placed in the ear canal;
a processor connected to the acoustic sensor for receiving the measured acoustic signal; and
a feedback device connected to the processor, wherein the feedback device is configured to provide feedback stimuli when the feedback device receives a bruxism event signal from the processor,
   wherein the processor is configured to
determine when a bruxism event occurs by comparing the acoustic signal, e.g. in a time-frequency-amplitude domain, with a target acoustic signal, e.g. in a time-frequency-amplitude domain;
send the bruxism event signal to the feedback device when the processor determines the occurrence of the bruxism event.

The use of the acoustic sensor allows for obtaining information from acoustic waves in the ear canal. Acoustic waves of the ear canal may originate due to jaw movements, e.g. due to bruxism, but also due to breathing, heart rate, or other contractions of muscles close to the ear canal. Thus the second aspect of the invention allows for an improved detection of the bruxism event by allowing for more information to be gathered by the sensor and by allowing this information to be used by the processor in determining the occurrence of the bruxism event.

By measuring the acoustic signal, not only may the system use information related to the change in shape of the ear canal, the system may also use other information available in the signal. For example, this allows for the use of Al techniques wherein detection of a bruxism event is learned by the processor based on all available data. This may lead to a personalized bruxism detection and feedback system wherein the system uses a personalized target characteristic based on all available data for the determining of the occurrence of a bruxism event.

According to the second aspect of the invention, instead of the acoustic sensor, the bruxism detection and feedback system may also comprise an accelerometer for measuring a movement signal and providing the signal to the processor. The processor in this case is configured to determine the bruxism event by comparing the measured movement signal to a target movement signal.

In embodiments of the second aspect of the invention the bruxism detection and feedback system further comprises a heart rate sensor and/or breathing rate sensor to measure a heart rate and/or breathing rate and wherein the processor is configured to determine the occurrence of a microarousal event based on the heart rate and/or breathing rate. The heart rate sensor and/or the breathing rate sensor may be formed by the acoustic sensor.

In further embodiments of the second aspect of the invention the processor is further configured to increase a measurement rate of the acoustic sensor when the occurrence of the microarousal event is determined and to determine the occurrence of the bruxism event by comparing the characteristic signal measured at the increased measuring rate to a target characteristic.

In embodiments of the second aspect of the invention the in-ear device further comprises a flexible resonator provided in the in-ear portion, wherein the resonator is configured to vibrate at a resonance frequency when a resonance sound is emitted, wherein the resonator is connected to the in-ear portion such that, upon a change of shape of the ear canal, the resonance frequency of the resonator changes, e.g. by a deformation of the resonator, wherein the in-ear device further comprises a sound emitter for an excitation signal to excite the resonator, and wherein the acoustic sensor is configured to measure a vibration of the resonator caused by the excitation signal.

In embodiments of the second aspect of the invention in-ear device further comprises a sealing portion to seal the ear canal when the in-ear portion is placed in the ear canal.

In embodiments of the second aspect of the invention the in-ear portion comprises an impermeable pouch that conforms to a shape of the ear canal and wherein the acoustic sensor is provided in the impermeable pouch for measuring the amplitude and the frequency of the acoustic signal in a medium filling the pouch.

In further embodiments of the second aspect of the invention the pouch is filled with an incompressible fluid.

In embodiments of the second aspect of the invention the in-ear device is personalized such that the in-ear portion extends to the second bend of the ear canal when the in-ear portion is placed in the ear canal.

In embodiments of the second aspect of the invention the processor is further configured to determine when the bruxism event terminates, for example, by determining when the amplitude and/or the frequency of the acoustic signal stop coinciding with the target amplitude and/or the target frequency of the target acoustic signal.

In embodiments of the second aspect of the invention the in-ear portion is at least partially made from a shape conforming material and wherein the shape conforming material is one of a body heat-activated memory foam and a polymer, e.g. a moldable silicone.

In embodiments of the second aspect of the invention an intensity, frequency, pattern, and/or type of the feedback stimuli is determined by using a reinforcement learning algorithm.

In embodiments of the second aspect of the invention the feedback stimulus is one or more of a vibration, an acoustic signal, a mechanical stimulus and an electrical stimulus.

### BRIEF DESCRIPTION OF THE DRAWINGS

The invention will be explained below with reference to the drawing in which:

FIG. 1 shows a schematic depiction of a bruxism detection and feedback system;

FIG. 2 shows a first embodiment of an in-ear device provided in an ear canal;

FIG. 3 shows a second embodiment of an in-ear device provided in an ear canal;

FIG. 4 shows a third embodiment of an in-ear device provided in an ear canal; and

FIG. 5 shows a fourth embodiment of an in-ear device provided in an ear canal.

### DESCRIPTION OF EMBODIMENTS

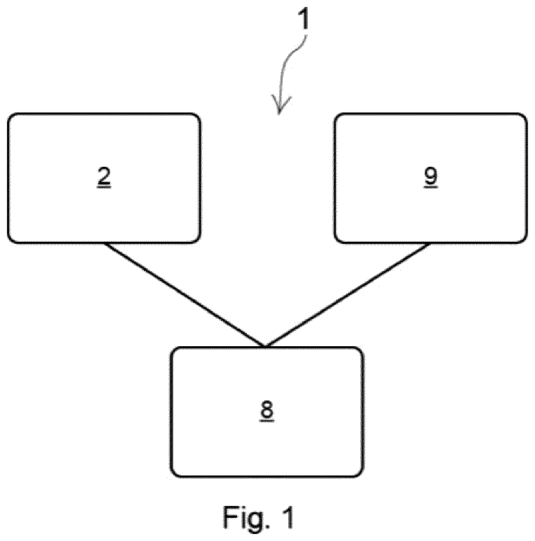

FIG. 1 shows a schematic depiction of a bruxism detection and feedback system 1 comprising an in-ear device 2 comprising a heart rate sensor 5 and/r or a breathing rate sensor 6 connected to a processor 8 and further comprising a feedback device 9. The feedback device 9 is connected to the processor 8. The processor 8 is further connected to the bruxism sensor 7 for measuring a characteristic signal of a bruxism event in the ear canal 4.

The processor 8 is configured to send the bruxism event signal to the feedback device 9 when the processor 8 determines the occurrence of the bruxism event. For example, the processor 8 may send multiple bruxism event signals when the feedback sent by the feedback device 9 has not stopped the bruxism event. The feedback stimuli may depend on an intensity of the bruxism event.

The processor 8 may be provided in the in-ear device 2, the feedback device 9, or in a separate device, such as a smart device, such as a smart phone or a smart watch.

The feedback device 9 may be embodied as a bracelet comprising a system for providing the feedback stimulus. The feedback device 9 may also be provided in the in-ear device 2 and for example provide an audible feedback stimulus.

The processor 8 is configured to
determine the occurrence of a microarousal event by comparing the measured heart rate, microvibrations and/or the measured breathing rate to a target heart rate and/or a target breathing rate;
increase a measuring rate of the bruxism sensor 7 when the occurrence of the microarousal event is determined by the processor 8;
receive the characteristic signal measured by the bruxism sensor 7;
determine the occurrence of a bruxism event by comparing the characteristic signal measured at the increased measuring rate to a target characteristic; and
send the bruxism event signal to the feedback device 9 when the processor determines the occurrence of the bruxism event.

This allows the processor 8 to switch the bruxism detection and feedback system 1 from a standby mode, wherein the measurement rate of the bruxism sensor 7 is lower, to an awake mode, wherein the measurement rate of the bruxism sensor 7 is higher. This allows for a more power efficient bruxism detection and feedback system 1 by reducing the necessary measurements of the bruxism sensor 7.

The processor 8 shown in FIG. 1 is further configured to determine when a bruxism event terminates, for example by determining when the characteristic signal stops coinciding with the target characteristic, or by determining when the characteristic signal drops below the target characteristic or when the characteristic signal no longer has a certain correlation to the target characteristic. This allows the processor 8 to send a signal to the feedback device 9 to stop providing feedback. This may further allow the processor 9 to reduce the measurement rate of the bruxism sensor 7, e.g. to a measurement rate before the occurrence of the microarousal event was determined, or e.g. such that the system 1 returns to a standby mode.

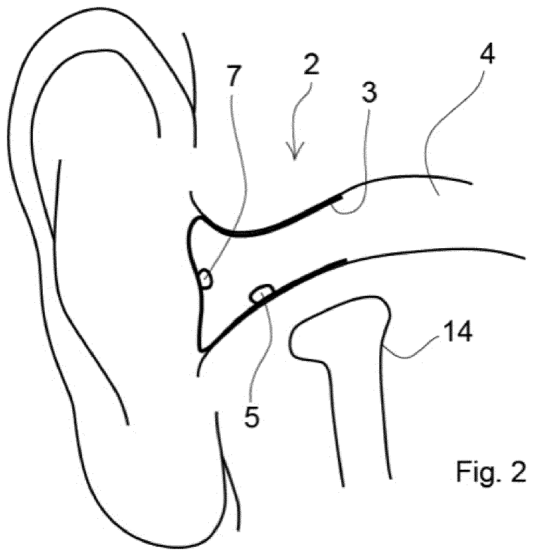

The in-ear device may be embodied in various different ways, for example as shown in FIGS. 2-5.

FIG. 2 shows a first embodiment of an in-ear device 2 provided in an ear canal 4. The ear canal 4 may be deformed by movement of the TMJ 14. The TMJ 14 may move as a result of jaw movement, such as chewing or clenching. The type of jaw movement translates into a correlated movement of the TMJ 14 which gives a correlated deformation, in particular a shape deformation, of the ear canal 4. By measuring the change in shape of the ear canal 4 the bruxism detection and feedback system 1 may determine if a bruxism event is occurring.

The first embodiment shown in FIG. 2 of the in-ear device 2 comprises an in-ear portion 3 which may be partly formed of a shape conforming material such as a memory foam or a moldable polymer. The in-ear portion 3 extends into the ear canal 4 and may react to changes in shape of the ear-canal. A heart rate sensor 5 is provided in the in-ear device 2 which measures the heart rate of the user, e.g. by monitoring a blood flow near the ear canal 4.

The first embodiment further comprises a bruxism sensor 7, embodied as an acoustic sensor, which measures a change of shape of the ear canal 4 via acoustic signals in the ear canal 4. In another example, the bruxism sensor 7 may be a strain gauge which comprises a wire grid provided in the in-ear portion 3 of the in-ear device 2. Changes in the shape of the in-ear portion 3 caused by changes in the shape of the ear canal 4 change the resistances of the wire grid which may be measured. In another example, the bruxism sensor 7 comprises magnets embedded in the in-ear portion 3 to create a known electromagnetic (EM) field in the ear canal 4 that changes upon changes in shape of the ear canal 4. Other embodiments of the bruxism sensor 7 are possible such as an optical sensor or a piezoelectric sensor.

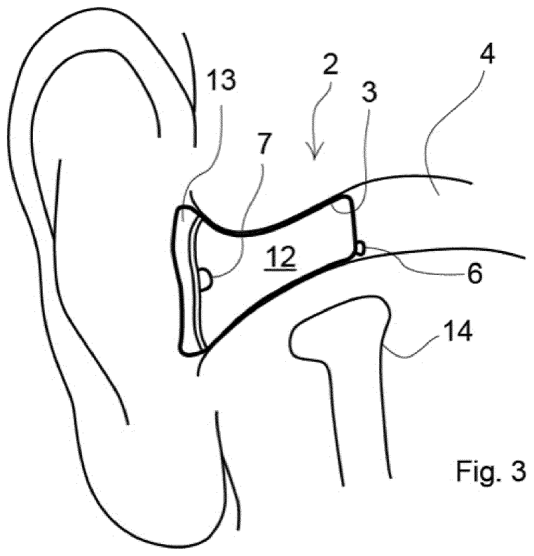

FIG. 3 shows a second embodiment of the in-ear device 2 provided in an ear canal 4. In this embodiment the in-ear portion 3 comprises an impermeable pouch 12 which is filled with an impermeable fluid. The pouch 12 conforms to the shape of the ear canal 4. Changes in the shape of the ear canal 4 are translated into signals in the fluid in the pouch which signals are measured by the bruxism sensor 7. The bruxism sensor 7 sends the measured signals to the processor 8 which may compare the signals to a target signal to determine whether or not a bruxism event is occurring.

The in-ear device 2 further comprises a breathing rate sensor 6 which measures a breathing rate of the user, e.g. by measuring an air flow in the ear canal 4 as a result of air flow in the oral and sinus cavities.

The in-ear device 2 further comprises a sealing portion 13 which seals the ear canal 4. This is particularly advantageous for measuring acoustic signals in the ear canal 4 because the acoustic signals are less interfered by outside noise.

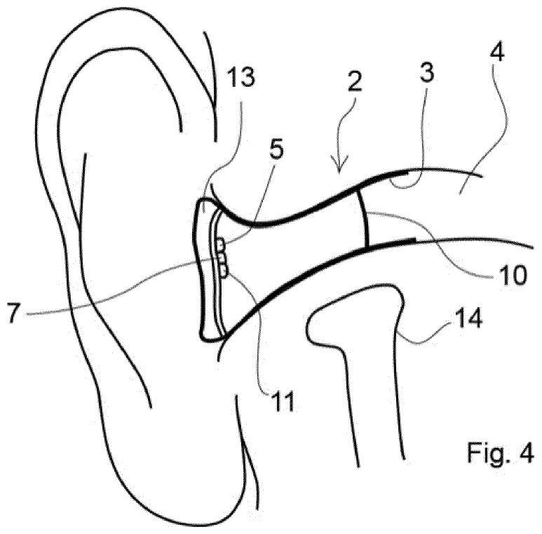

FIG. 4 shows a third embodiment of the in-ear device 2 provided in an ear canal 4. In this embodiment the in-ear device 2 comprises a flexible resonator 10 which is provided in the in-ear portion 3 of the in-ear device 2. The resonator 10 is configured to vibrate at a resonance frequency when a resonance sound is emitted. The resonator is connected to the in-ear portion such that, upon a change in shape of the in-ear portion 3, the resonance frequency of the resonator 10 changes, e.g. by a deformation of the resonator 10. The resonator 10 may be a flexible strand of material such as a string. Thus the resonance frequency depends on the shape of the ear-canal 4. The change of frequency of the vibration as a result of a fixed excitation signal is proportional to the amount of deformation of the ear canal 4.

The in-ear device 2 further comprises a sound emitter 11 for emitting an excitation signal for exciting the resonator 10. When the excitation signal has a frequency close to or at the current resonance frequency of the resonator 10 the resonator will start to vibrate, which vibration may be measured by the bruxism sensor 7. In the figure the bruxism sensor 7 may be an acoustic sensor, however in other embodiments the bruxism sensor 7 may be placed at or near to the resonator 10 to directly, e.g. through contact with the resonator 10, measure vibrations of the resonator 10.

The excitation signal may be a periodic discrete signal at a fixed frequency, but it may also be a periodic continuous signal over a frequency range, or even a non-periodic signal. The vibration may be measured by an acoustic sensor which hears the acoustic signal emitted by the resonator. The vibration may also be measured by a force transducer provided near or at the resonator 10.

The resonator 10 may be formed by multiple flexible strands of material, e.g. by multiple strings, with different orientation to increase the information. The resonator 10 may comprise multiple resonators having different resonator frequencies or located along different points of the in-ear portion 3 to provide information on changes in shape of different portions of the ear canal 4. The resonator 10 may further be a ring resonator having a ring like structure or a diaphragm resonator having a closed circle structure.

The in-ear device 2 further comprises a sealing portion to seal the ear canal 4 to reduce outside influence on the measurements performed by the in-ear device.

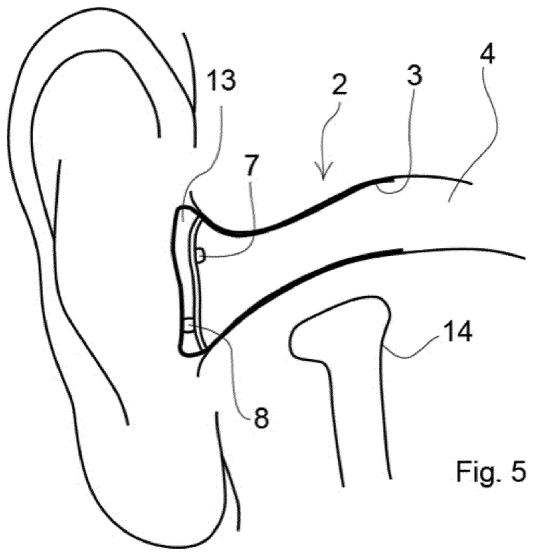

FIG. 5 shows a fourth embodiment of the in-ear device 2 in the ear canal 4 according to the second aspect of the invention. In this embodiment the bruxism sensor 7 is embodied as an acoustic sensor for measuring acoustic signals, e.g. acoustic waves, in the ear canal 4 which are the result of changes of shape of the ear canal 4.

The use of the acoustic sensor allows for obtaining information from acoustic waves in the ear canal 4. Acoustic waves of the ear canal 4 may originate due to jaw movements, e.g. due to bruxism, but also due to breathing, heart rate, or other contractions of muscles close to the ear canal. Thus the second aspect of the invention allows for an improved detection of the bruxism event by allowing for more information to be gathered by the bruxism sensor 7 and by allowing this information to be used by the processor 8 in determining the occurrence of the bruxism event.

By measuring the acoustic signal, not only may the system use information related to the change in shape of the ear canal, the system may also use other information available in the signal. For example, this allows for the use of Al techniques wherein detection of a bruxism event is learned by the processor based on all available data. This may lead to a personalized bruxism detection and feedback system wherein the system uses a personalized target characteristic based on all available data for the determining of the occurrence of a bruxism event.

The in-ear device 2 is further provided with a sealing portion 13 which, in this case, comprises the processor 8 connected to the bruxism sensor 7 and to the feedback device 9.

1. A bruxism detection and feedback system comprising:
an in-ear device comprising:
an in-ear portion configured to be placed in an ear canal, wherein preferably the in-ear portion is at least partially made from a shape conforming material that conforms to a shape of the ear canal;
a heart rate sensor to measure a heart rate, a microvibration sensor to measure microvibrations and/or a breathing rate sensor to measure a breathing rate when the in-ear portion is placed in the ear canal; and
a bruxism sensor configured for measuring a characteristic signal which is characteristic of a bruxism event when the in-ear portion is placed in the ear canal,
a processor connected to the heart rate sensor, the microvibration sensor and/or the breathing rate sensor for receiving the measured heart rate, microvibrations, and/or measured breathing rate, and connected to the bruxism sensor for receiving the measured characteristic signal; and
a feedback device connected to the processor, wherein the feedback device is configured to provide a feedback stimulus when the feedback device receives a bruxism event signal from the processor,
wherein the processor is configured to
determine the occurrence of a microarousal event by comparing the measured heart rate, the measured microvibrations and/or the measured breathing rate to a target heart rate and/or a target breathing rate;
increase a measuring rate of the bruxism sensor when the occurrence of the microarousal event is determined by the processor;
receive the characteristic signal measured by the bruxism sensor;
determine the occurrence of a bruxism event by comparing the characteristic signal measured at the increased measuring rate to a target characteristic; and
send the bruxism event signal to the feedback device when the processor determines the occurrence of the bruxism event.

2. The bruxism detection and feedback system according to claim 1, wherein the bruxism sensor is an acoustic sensor for measuring an acoustic signal, when the in-ear portion is placed in the ear canal,
wherein the occurrence of the bruxism event by the processor is determined by comparing the measured acoustic signal to a target acoustic signal.

3. The bruxism detection and feedback system according to claim 2, wherein the heart rate sensor and/or the breathing rate sensor are formed by the acoustic sensor.

4. The bruxism detection and feedback system according to claim 1, wherein the in-ear device further comprises a flexible resonator provided in the in-ear portion, wherein the resonator is configured to vibrate at a resonance frequency when a resonance sound is emitted,
wherein the resonator is connected to the in-ear portion such that, upon a change of shape of the ear canal, the resonance frequency of the resonator changes,
wherein the in-ear device further comprises a sound emitter for emitting an excitation signal to excite the resonator, and wherein the bruxism sensor is configured to measure a vibration of the resonator caused by the excitation signal.

5. The bruxism detection and feedback system according to claim 1, wherein the in-ear device further comprises a sealing portion to seal the ear canal when the in-ear portion is placed in the ear canal.

6. The bruxism detection and feedback system according to claim 1, wherein the in-ear portion comprises an impermeable pouch that conforms to a shape of the ear canal and wherein the bruxism sensor is provided in the impermeable pouch for measuring the characteristic signal in a medium filling the pouch.

7. The bruxism detection and feedback system according to claim 6, wherein the pouch is filled with an incompressible fluid.

8. The bruxism detection and feedback system according to claim 1, wherein the in-ear device is personalized such that the in-ear portion extends to the second bend of the ear canal when the in-ear portion is placed in the ear canal-.

9. The bruxism detection and feedback system according to claim 1, wherein the processor is further configured to determine when the bruxism event terminates.

10. The bruxism detection and feedback system according to claim 1, wherein the in-ear portion is at least partially made from a shape conforming material and wherein the shape conforming material is one of a body heat-activated memory foam or a silicone.

11. The bruxism detection and feedback system according to claim 1, wherein the system is configured to determine an intensity, frequency and/or type of the feedback stimulus by using a reinforcement learning algorithm.

12. The bruxism detection and feedback system according to claim 1, wherein the feedback stimulus is one or more of a haptic feedback, an acoustic signal, a mechanical stimulus and an electrical stimulus.

13. The bruxism detection and feedback system according to claim 1, wherein the processor is further configured to determine a type of bruxism event and wherein the processor is configured to determine a type of feedback stimulus based on the type of the bruxism event.

14. A method for detecting of bruxism wherein use is made of a bruxism detection and feedback system according to claim 1.

15. The method according to claim 14, wherein the method comprises:
placing the in-ear portion in the ear canal;
measuring a heart rate using the heart rate sensor, microvibrations using the microvibration sensor and/or a breathing rate using the breathing rate sensor;
determining, by the processor, the occurrence of a microarousal event by comparing the measured heart rate, the measured microvibrations and/or the measured breathing rate to the target heart rate, target microvibrations and/or the target breathing rate;
increasing a measuring rate of the bruxism sensor when the occurrence of the microarousal event is determined by the processor;
receiving a characteristic signal measured by the bruxism sensor; and
determining the occurrence of a bruxism event by comparing the characteristic signal measured at the increased measuring rate to a target characteristic.

16. The method according to claim 14, wherein the method comprises:
placing the in-ear portion in the ear canal;
measuring the characteristic signal using the bruxism sensor;
determining, by the processor, the occurrence of the bruxism event by comparing the characteristic signal to the target characteristic;
sending, by the processor, the bruxism event signal to the feedback device when the processor determines the occurrence of the bruxism event; and
providing a feedback stimulus by the feedback device.
